(19) United States
(12) Patent Application Publication
(10) Pub. No.: US 2013/0231262 A1
(43) Pub. Date: Sep. 5, 2013

(54) ENGINEERED PROTEIN: "2-COLOR SERCA", AN ION-MOTIVE ATPase FUSED TO CERULEAN AND YELLOW FLUORESCENT PROTEIN

(71) Applicant: LOYOLA UNIVERSITY CHICAGO, Maywood, IL (US)

(72) Inventor: Seth L. Robia, Westchester, IL (US)

(73) Assignee: Loyola University Chicago, Maywood, IL (US)

(21) Appl. No.: 13/785,837

(22) Filed: Mar. 5, 2013

Related U.S. Application Data
(60) Provisional application No. 61/606,647, filed on Mar. 5, 2012.

(51) Int. Cl. G01N 33/68; C12Q 1/34; C12N 9/14

(52) U.S. Cl. CPC G01N 33/6887; C12N 9/14; C12Q 1/34
USPC 506/9; 435/195; 435/18; 506/11

(57) ABSTRACT

A method and engineered proteins for use therewith suitable for studying SERCA that are capable of being used in vivo and do not require protein purification or chemical labeling of SERCA, or reconstitution into artificial membranes. The engineered protein for calcium handling within human cells includes a two-color SERCA construct having three component proteins fused together. The three component proteins include a blue fluorescent protein (cerulean), SERCA2a and a yellow fluorescent protein (YFP), or a red fluorescent protein (tagRFP acceptor), SERCA and a green fluorescent protein (GFP). The method of determining SERCA activity for optimization of cardiac function includes resolving structure changes of the two-color SERCA construct. The two-color SERCA constructs are catalytically active and able to pump calcium following the step of resolving structure changes.

### CROSS REFERENCE TO RELATED APPLICATIONS

This application claims the benefit of U.S. Provisional Application No. 61/606,647, filed Mar. 5, 2012, the contents of which are incorporated herein by reference.

### BACKGROUND OF THE INVENTION

The present invention generally relates to engineered proteins. More particularly, this invention relates to an engineered protein for calcium handling within human cells, the determination of SERCA activity for optimization of cardiac function, and the potential for pharmacological treatment.

The sarco(endo)plasmic reticulum calcium adenosine triphosphatase (SERCA) is the ion-motive ATPase responsible for maintaining the 7,000 fold Ca gradient across the membrane of the endoplasmic reticulum (ER). Specifically, SERCA is a P-type ion pump responsible for transporting Ca ions from the cytoplasm to sarcoendoplasmic reticulum (SR) in muscle cells.

SERCA plays a particularly important role in striated muscle, where Ca release and reuptake determines the contraction and relaxation of the muscle. This process is critical during cardiac diastole (relaxation), as the muscle must relax to allow for adequate filling of the ventricles with blood. SERCA function is also important for systole (cardiac contraction), as it is solely responsible for creating the large store of SR Ca that is released to initiate activation of the muscle.

In particular, optimal calcium handling is critical for normal cardiac function; deranged SERCA activity has been implicated as a cause and an effect of heart failure. This and other P-type ATPases are considered high value targets for pharmacological treatment of heart failure. Prior investigations into SERCA have used optical methods to study the dynamic transitions of SERCA protein structure. These studies generally used extrinsic fluorescent probes.

Traditional fluorescent labeling schemes require thiol- or amine-reactive chemistries, some of which are known to inactivate ATPases. For example, a known hybrid approach utilizes a CFP-SERCA labeled with a fluorescein dye. All such assays require detergent solubilization and purification of proteins, and reconstitution into artificial membranes. Such steps risk compromising protein function. A significant disadvantage associated with these conventional methods is that they are not compatible with live cell experiments.

In view of the above, it can be appreciated that it would be desirable if improved methods were available for studying SERCA that were capable of being used in vivo and do not require protein purification or chemical labeling of SERCA, or reconstitution into artificial membranes.

### BRIEF DESCRIPTION OF THE INVENTION

The present invention provides a method and engineered proteins for use therewith suitable for studying SERCA that are capable of being used in vivo and do not require protein purification or chemical labeling of SERCA, or reconstitution into artificial membranes.

According to a first aspect of the invention, an engineered protein for calcium handling within human cells includes a two-color SERCA construct comprising three component proteins fused together. The three component proteins comprise a blue fluorescent protein (cerulean donor), SERCA2a and a yellow fluorescent protein (YFP acceptor), or a red fluorescent protein (tagRFP acceptor), SERCA and a green fluorescent protein (GFP donor).

According to a second aspect of the invention, a method of determining SERCA activity for optimization of cardiac function includes resolving structure changes of a two-color SERCA construct comprising three component proteins fused together. The two-color SERCA construct is catalytically active and able to pump calcium following the step of resolving structure changes.

A technical effect of the invention is the ability to study the function and structure of SERCA in vivo. In particular, it is believed that, by forming two-color SERCA constructs comprising fluorescent proteins, the engineered proteins will remain catalytically active and able to pump calcium following the step of resolving structure changes, and therefore SERCA can be studied during its normal function.

Other aspects and advantages of this invention will be better appreciated from the following detailed description.

### BRIEF DESCRIPTION OF THE DRAWINGS

FIG. 1 includes two perspective views representing possible conformational changes of SERCA in response to Ca binding. (A) Opening of the cytoplasmic headpiece, increasing N- to A-domain distance. (B) Closure of the cytoplasmic headpiece, decreasing domain separation. The cleft between the N- and A-domains is highlighted with dotted lines.

FIG. 2 includes a schematic diagram and a perspective view representing 2-color SERCA constructs in accordance with an aspect of this invention. (A) Schematic diagram of construct 509 including a 2-color SERCA comprising SERCA2a with an N-terminal Cer and an intrasequence YFP. (B) Fusion positions for Cer (N-term) and intrasequence YFP (509, 576, 661, or C-term).

FIG. 3 is a set of graphs representing Ca uptake in live cells quantified from changes in cytosolic Ca measured by X-rhod 1 fluorescence. Application of extracellular ATP elicited an increase in cytosolic Ca in untransfected cells (black). This Ca transient was largely abolished in cells in the same microscopic field that expressed 2-color SERCA constructs, suggesting Ca uptake by 2-color SERCA. Coexpression of PLB partially restored the ATP-releasable Ca transient, consistent with inhibition of 2-color SERCA by PLB. The Ca content of the ER was evaluated by application of 10 mM Tg. Cells expressing 2-color SERCA had a larger Tg-releasable ER content compared to untransfected cells (black). This difference was abolished by coexpression of PLB. (A) Uptake of Ca by 509. (B) Summary of 509 uptake (normalized area under the peak). (C) 576. (D) Summary of 576. (E) 661. (F) Summary of 661. (G) C-term. (H) Summary of C-term. (*) indicates 2-color SERCA was significantly different from paired control (untransfected cells). (#) indicates 2-color SERCA+PLB was significantly different from unpaired 2-color SERCA alone.

FIG. 4 is a graph representing calcium-dependent ATPase activity of Cer-SERCA2a and 2-color SERCA (construct 509) measured in cell homogenates. The Ca-sensitivity was indistinguishable from that of skeletal SR and greater than that of cardiac SR (black), presumably due to the presence of phospholamban in the latter. The specific ATPase activity (ATP hydrolyzed per s per SERCA2a at 37 degrees C.) at saturating Ca2+(pCa 5.0) was indistinguishable for cardiac SR, Cer-SERCA2a, and 2-color SERCA.

FIG. 5 are scanned images representing TIRF microscopy of AAV-293 cells expressing 2-color SERCA (509). Fluorescence was distributed in a reticulated pattern consistent with ER localization.

FIG. 6 includes scanned images and graphs representing characterization of 2-color SERCA. (A) Confocal microscopy showed 2-color SERCA was localized in internal perinuclear membranes. Plasma membranes were counterstained with FM 4-64. (B) 2-color SERCA showed concentration-independent intramolecular FRET (black points, dotted line). A hyperbolic dependence on protein expression was observed for intermolecular FRET from Cer-SERCA to YFP-PLB (grey points, solid line). (C) The brightness ratio Cer/YFP sensitivity ratio for microscopy setup was 0.5 (vertical line). Deviation from this value suggests a Cer/YFP stoichiometry other than 1:1. C32V and VCV are controls with 1:1 and 1:2 stoichiometry, respectively.

FIG. 7 includes graphs representing dynamic FRET changes of SERCA constructs expressed in AAV-293 cells. Data represent control intact cells, cells permeabilized with ionophore with 5 mM EGTA or 2 mM Ca in the extracellular solution, or intact cells treated with thapsigargin.

FIG. 8 includes graphs representing Tg-dependent conformational changes of 2-color SERCA (construct 509). (A) The rate of change of FRET was Tg concentration dependent. (B) Tg titration of 2-color SERCA, measured at different time points. (C) The time dependent shift in apparent affinity of SERCA for Tg. (D) The rate of Tg binding was not changed by coexpression of PLB with 2-color SERCA compared to 2-color SERCA alone (black).

FIG. 9 includes graphs representing Ca2+-dependent conformational changes of 2-color SERCA (construct 509). (A) Fluorescence emission measured in the donor channel (DD) and acceptor channel (AA), and the acceptor emission with donor excitation (DA) after the addition of DMSO vehicle. (B) Fluorescence emission after addition of ionomycin in 2 mM Ca. (C) Fluorescence emission after addition of ionomycin in the presence of EGTA. (D) The ratio of DA and DD fluorescence intensities suggest that FRET is increased with Ca, and decreased with EGTA compared to DMSO vehicle. (E) The corresponding FRET efficiency calculated according to the E-FRET method.

FIG. 10 includes perspective views and graphs representing single molecule fluorescence spectroscopy of 2-color SERCA. (A) Cer-SERCA-YFP comprises a Cerulean FRET donor fused to the n-terminus of SERCA in the actuator (A) domain and an intrasequence YFP inserted in the nucleotide binding (N) domain. (B) RG-SERCA is an analogous construct composed of a tagRFP acceptor fused to the N-terminus in the A-domain and GFP donor inserted in the N-domain. Changes in SERCA conformation alter the distance between the N- and A-domains, changing intramolecular FRET efficiency. (C) Fluorescence bursts from single molecules of detergent-solubilized 2-color SERCA. (D) A histogram of the fluorescence lifetime measurements obtained from 2-color RG-SERCA or control GFPSERCA (black).

FIG. 11 includes graphs representing single molecule fluorescence spectroscopy of 2-color SERCA. (A) The histogram of FRET efficiency calculated from the lifetime measurements shown in FIG. 10D. A Gaussian fit revealed subpopulations consistent with four discrete conformations of SERCA. (B) Cer-SERCA-YFP also exhibited four discrete FRET states. The reduced FRET of these states (compared to FIG. 11A) was consistent with a shorter R₀ for the Cer-YFP FRET pair. (C) RG-SERCA single molecule FRET time trajectory. Horizontal lines indicate states I (lowest) through IV (highest) identified by Gaussian analysis (as in FIG. 11A). The pump is observed to transiently sample the low FRET state (at arrow), before returning to higher FRET conformations, suggesting that State I does not represent denatured protein. (D) Analysis of dwell time for SERCA sampling states I-IV revealed a biphasic distribution of dwell times characterized by fast (80 μs) and slow (690 μs) kinetics.

FIG. 12 includes scanned images and graphs representing 2-color SERCA expressed in adult ventricular cardiac myocytes. (A) Localization of RG-SERCA GFP fluorescence in adult ventricular myocytes. (B) Localization of RG-SERCA tagRFP fluorescence. (C) FRET distribution of 2-color SERCA during relaxation (diastole). (D) Systole. (E) Diastole, +isoproteronol. (F) Systole, +isoproteronol. FIGS. 12C-12F are representative data obtained from individual cells.

FIG. 13 includes graphs representing 2-color SERCA expressed in adult ventricular cardiac myocytes. (A) Summary of FRET subpopulations during diastole before (solid bars) or after addition of isoproterenol (striped bars). (B) Systole, before (solid bars) or after addition of isoproterenol (striped bars). (C) FRET distribution after addition of Tg. (D) Summary of C. FIG. 13C is representative data obtained from an individual cell. FIGS. 13A, 13B, 13D are mean±SE of all data.

FIG. 14 includes scanned images and graphs representing expression of 2-color SERCA in AAV-293 cells. (A) Localization of RG-SERCA GFP fluorescence in AAV-293 cells. (B) Localization of RG-SERCA tagRFP fluorescence. (C) FRET distribution of 2-color SERCA and non-phosphorylatable PLB in intact cells. (D) As in B, +ionophore. (E) 2-color SERCA+phosphomimetic PLB. (F) As in D, +ionophore. FIGS. 14C-14F are representative data obtained from individual cells.

FIG. 15 includes graphs representing expression of 2-color SERCA in AAV-293 cells. (A) Summary of FRET subpopulations from intact cells expressing 2-color SERCA and non phosphorylatable PLB (solid bars) or phosphomimetic PLB (striped bars). (B)+ionophore, with nonphosphorylatable PLB (solid bars) or phosphomimetic PLB (striped bars). (C) FRET distribution after addition of Tg. (D) Summary of C. FIG. 15C is representative data obtained from an individual cell. FIGS. 15A, 15B, 15D are mean±SE of all data.

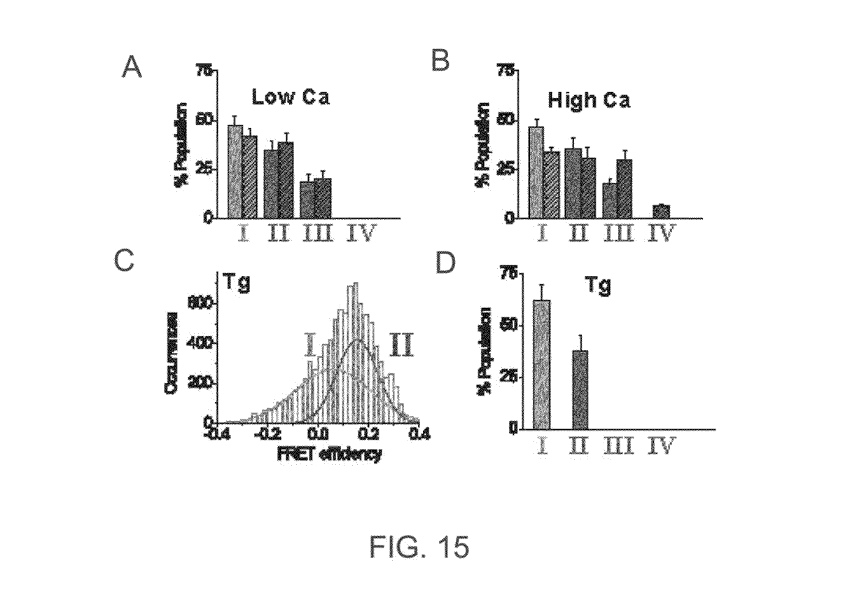

FIG. 16 includes graphs and perspective views representing comparison of FRET histogram Gaussian fit width vs. center position data obtained from cardiac myocytes. (A) States I-IV for RG-SERCA showed that the population Gaussian width decreased with increasing population Gaussian center value. This relationship was not detectably altered by Tg (triangles) or iso (squares) compared to control (circles). The data suggest that low FRET (open) conformations are more dynamically disordered than high FRET (closed) structures. (B) Average RG-SERCA peak parameters (black) compared with Cer-SERCA-YFP. The shorter R₀ of the Cer/YFP pair yielded decreased FRET (decreased center values), but similar Gaussian widths for each state. Data are mean±SE. (C) Proposed novel conformation of SERCA: “open” E2 (Ca-free) SERCA, characterized by a dynamically disordered cytoplasmic headpiece. (D) Proposed “closed” E1 (Ca-bound) SERCA, with a rigidly ordered headpiece. The conformation represents a unique E1 state that is stabilized by phosphorylated phospholamban.

FIG. 17 is a graph representing the fluorescence lifetime measurements obtained from Cer-SERCA-YFP or control Cer-SERCA (black).

FIG. 18 includes scanned images and graphs representing Cer-SERCA-YFP expressed in cardiac myocytes. (A) Cer-SERCA-YFP Cer fluorescence was localized in striations and longitudinal stripes. (B) Cer-SERCA-YFP YFP fluorescence. (C) FRET distribution of 2-color SERCA during relaxation (diastole). (D) Systole. (E) Diastole, +isoproteronol. (F) Systole, +isoproteronol.

FIG. 19 includes graphs representing changes in the average population of FRET substates in a cardiac myocyte during electrical pacing at 0.25 Hz after stimulation with isoproterenol. (A) E1 substate population changes are shown as stacked lines, where black squares represent FRET State III and circles represent the sum of States III and IV. The onset of myocyte contraction is indicated (arrow). (B) E2 substate population changes, where black squares represent State I and circles represent the sum of I and II. The data are assembled from 16 consecutive contractions, gated with 100 ms bin resolution. Overall, the data show that E1 states accumulate during systole at the expense of E2 states.

### DETAILED DESCRIPTION OF THE INVENTION

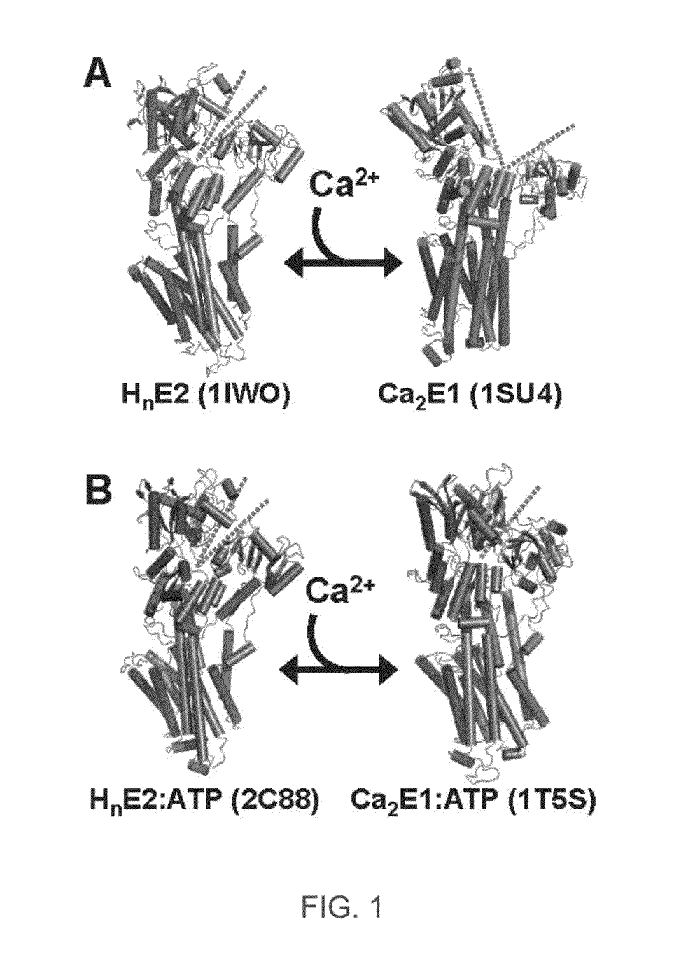

The present invention is generally applicable to methods and assays for studying the structure and function of SERCA. SERCA is believed to undergo conformational changes during catalytic cycling. In particular, X-ray crystallography have provided many atomic resolution SERCA structures suggesting that the relative motions of SERCA cytoplasmic domains result in opening and closing of the cytoplasmic headpiece during the transition between the E1 (Ca-bound) and E2 (Ca-free) enzymatic substrate (FIG. 1).

The E1 (Ca bound) and E2 (Ca free) conformations are characterized by different relative positions of three cytoplasmic domains (N, A, P), collectively known as the cytoplasmic headpiece. Conformational changes in the headpiece are transmitted to the trans-membrane (TM) domain to alter the occlusion and affinity of the Ca binding sites. The first X-ray crystal structures of SERCA suggested that Ca induces a large increase in the separation of the N- and A-domains, but recent crystallography and in vitro fluorescence resonance energy transfer (FRET) experiments have depicted a closed cytoplasmic headpiece for Ca-bound SERCA, and suggest that the conformational change may be smaller than previously thought.

FIGS. 1A and 1B represent two possible changes of SERCA in response to Ca binding. The cleft between the N- and A- is highlighted with dots. FIG. 1A represents a large distance of the cytoplasmic headpiece between the N- and A-domains which has previously been believed to be an open position of the cytoplasmic headpiece. FIG. 1B represents a smaller distance of the cytoplasmic headpiece between the N- and A-domains which has previously been believed to be a closed position of the cytoplasmic headpiece. A challenge for interpreting these high-resolution structures is that functionally important conformations may be overlooked by X-ray crystallography, which is biased toward compact conformations and well-ordered structures.

To directly quantify SERCA headpiece conformational changes in live cells, the present invention provides methods of measuring intramolecular fluorescence resonance energy transfer (FRET) between fluorescent protein fusion tags directed to various sites on the SERCA cytoplasmic domains. The methods utilize engineered SERCA proteins to determine how the domain separation distance changes with Ca binding. Unlike prior methods of studying SERCA, the methods described herein takes advantage of the intrasequence tag, which can be fused to sites on SERCA. In addition, the fluorescent construct is entirely biosynthetic and compatible with live cell measurements. Therefore, unlike prior methods, the structure and function of SERCA can be studied in vivo. This is important since many drug candidates identified from in vitro screens later fail because they cannot penetrate cell membranes or because they are toxic to cells. This is not identified in the first screen, only in subsequent follow-up permeability/toxicity screens in cells or in live animals. This may add significantly to the cost of screening. 2-color SERCA avoids this by combining the primary optical screen together with a live cell screen, all together in a single step. By quantifying relative changes in probe separation distance, it is believed that insight can be gained into the conformational changes of the SERCA cytoplasmic headpiece.

According to an aspect of the invention, engineered proteins were produced to report changes in the conformation of the SERCA. The proteins are a two-color SERCA construct comprising three component proteins fused together. Examples of the three component proteins include a blue fluorescent protein (cerulean donor), SERCA2a and a yellow fluorescent protein (YFP acceptor), or a red fluorescent protein (tagRFP acceptor), SERCA and a green fluorescent protein (GFP donor). Although the invention will be discussed hereinafter in reference to the above two examples, additional constructs are foreseeable. In addition, the methods described herein may be applicable to other embodiments, for example, constructs could be produced for the highly homologous sodium/potassium ATPase (NKA, particularly with regard to the cardiac environment), an ABC transporter (useful for screening drugs for cystic fibrosis, cancer, multi-drug resistance), skeletal muscle SERCA (useful for screening drugs for treatment of muscular dystrophy), or other SERCA species (useful for screening drugs for treatment of malaria). Each construct is self-contained, encoded by a single plasmid.

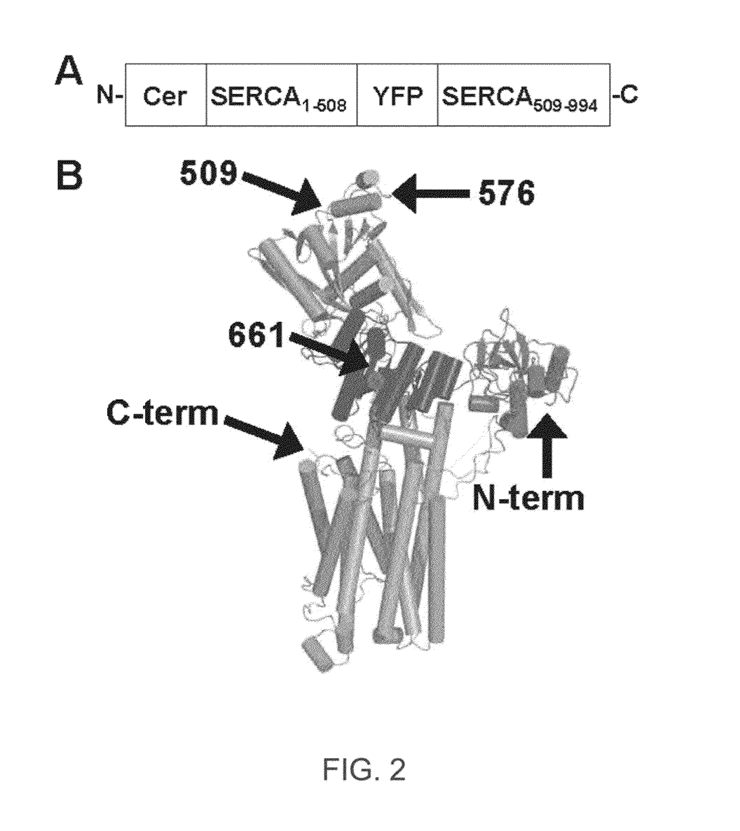

In a first set of experiments studying SERCA, mCerulean (Cer) was fused to the N-terminus of canine SERCA2a to form Cer-SERCA-YFP constructs. This fusion position is in the A-domain of the pump, represented in FIG. 2. YFP was fused as an intrasequence tag before residue 509 or 576 in the N-domain, or 661 in the P-domain, or at the SERCA C-terminus in the TM domain (FIG. 2B). Structures provided in FIG. 1 and FIG. 2 correspond to the isoform SERCA1a, which is 84% homologous to SERCA2a. Intrasequence fusion positions were chosen using E1/E2 x-ray crystal structures of SERCA1a to identify unstructured loops and predict large relative distance changes from E1 to E2. The gene encoding the fluorescent protein was inserted into the gene encoding the SERCA pump according to standard recombinant DNA techniques. A reading frame of the SERCA pump is the same as the fluorescent protein, so both are expressed together as a single polypeptide.

In producing the constructs, regions important for SERCA function were avoided. Fluorescence microscopy of AAV-293 cells (Agilent, Santa Clara, Calif.) expressing 2-color SERCA was performed in glass-bottom chambered coverslips (Matek Corporation, Ashland, Mass.) coated with poly-D-lysine (Sigma-Aldrich) 18-24 hours post-transfection with MBS mammalian transfection kit (Stratagene, La-Jolla, Calif.).

To determine the effect of Ca on the SERCA structure, the cells were permeabilized for 1 minute in 50 mM ionomycin in growth medium (90% Dulbecco's Modified Eagle Medium, 10% fetal bovine serum, 1% glutamine). To reduce intracellular Ca, the cells were permeabilized with 50 μM ionomycin in 5 mM EGTA in phosphate-buffered saline or relaxing solution (100 mM KCl, 5 mM EGTA, 5 mM MgCl2, 3.2 mM ATP, 10 mM imidazole pH=7.2). Confocal microscopy was performed using a Leica SP5 confocal microscope equipped with a 1.3 N.A. 63× water immersion objective. Excitation was accomplished with Ar laser illumination at 458 nm for Cer, 514 nm for YFP and FM 4-64, with emission bands of 467-512 nm for Cer, 528-565 for YFP, and 674-797 nm for FM 4-64, using sequential image acquisition. Total internal reflection fluorescence (TIRF) microscopy was performed with a Nikon inverted microscope (Ti-e) equipped with a 100× oil-immersion objective with (N.A.=1.49) and a cooled CCD camera (Coolsnap K4, Photometrics, Tucson, Ariz.). Through-objective TIRF excitation was achieved with a 449 nm diode laser (for Cer) or 514 nm Ar laser (for YFP). The laser incident angle was adjusted to create an evanescent field that illuminated the plasma membrane in contact with the surface substrate and a thin section of endoplasmic reticulum. Widefield lamp excitation was used for FRET measurements, using computer-controlled filter wheels, as known in the art. Fluorescent images were recorded with a cooled EM-CCD camera (Andor Ixon, Belfast, Northern Ireland). The RG-SERCA constructs in the second set of experiments discussed below were produced by substantially similar methods as the YFP constructs discussed above.

To test whether the Cer-SERCA-YFP constructs show structural substrate-dependent FRET, the steady state was measured in the presence and absence of thapsigargin and Calcium.

To evaluate Ca transport activity for 2-color SERCA constructs, a live-cell Ca uptake assay was performed. Heterogeneously transfected populations of AAV-293 cells were incubated with cell permeant Ca indicator dye X-rhod 1 (AM) (Invitrogen, Carlsbad, Calif.). Transfected and untransfected cells were distinguished on the basis of the intensity of YFP fluorescence emission. Release of Ca from intracellular stores was accomplished by stimulating purinergic receptors with extracellular application of 100 μM ATP. Accumulation of Ca in the cytosol was quantified as an increase in X-rhod 1 fluorescence, and was the net result of Ca release counterbalanced by Ca extrusion and uptake processes, including SERCA activity. Exogenous SERCA activity was detected as a decrease in ATP-stimulated cytosolic Ca accumulation relative to untransfected cells in the same microscopic field. Three minutes after application of extracellular ATP, cells were treated with 10 μM Tg to determine the size of the Ca store remaining in the ER. The magnitudes of the ATP- and Tg dependent Ca transients were quantified by integrating the area under the trace of X-rhod 1 fluorescence vs. time. The integrated area of Ca transients in 2-color SERCA transfected cells was compared to corresponding control (untransfected cells) using a paired t-test, with a p-value of greater than 0.05 taken to indicate a significant difference. To determine whether the 2-color SERCA samples were regulated by phospholamban (PLB), cells transfected with 2-color SERCA were compared to separate samples transfected with both 2-color SERCA and YFP-PLB using an unpaired t-test, with a p-value of greater than 0.05 taken to be a significant difference. For Cer-SERCA2a and construct 509, enzymatic activity was also quantified in cell homogenates by spectrophotometric measurement of the rate of NADH consumption in an enzyme-coupled activity assay. Conventional cell transfection did not yield adequate expression of SERCA protein for this assay, so AAV-293 cells were infected with adenoviruses encoding Cer-SERCA2a or 509 resulting in much greater protein expression, as quantified by immunoblotting.

FRET was quantified using the E-FRET (3-cube) method. FRET efficiency (E) was calculated according to the relationship

E=IDA−aIAA−d(IDD)IDA−aIAA+G−d(IDD)

where IDD is the intensity of fluorescence emission detected in the donor channel (472/30 nm) with excitation of 427/10 nm; IAA is acceptor channel (542/27 nm) emission with excitation of 504/12 nm; IDA is the “FRET” channel, with 542/27 nm emission and excitation of 427/10 nm; a and d are cross-talk coefficients determined from acceptor-only or donor-only samples, respectively. Values of d=0.7 (for Cer) and a=0.074 (for YFP) were obtained. G is the ratio of the sensitized emission to the corresponding amount of donor recovery, which was 3.2 for this set of experiments. Probe separation distance (R) was calculated from the relationship described by Förster, R=(R₀)[(1/E)−1)^{1/6}, where E is the measured FRET value and R₀ is the Förster radius, which is 49.8 Å for the Cer-YFP pair. E-FRET measurements were also compared with FRET obtained by the photobleaching method. All error bars represent mean±SE.

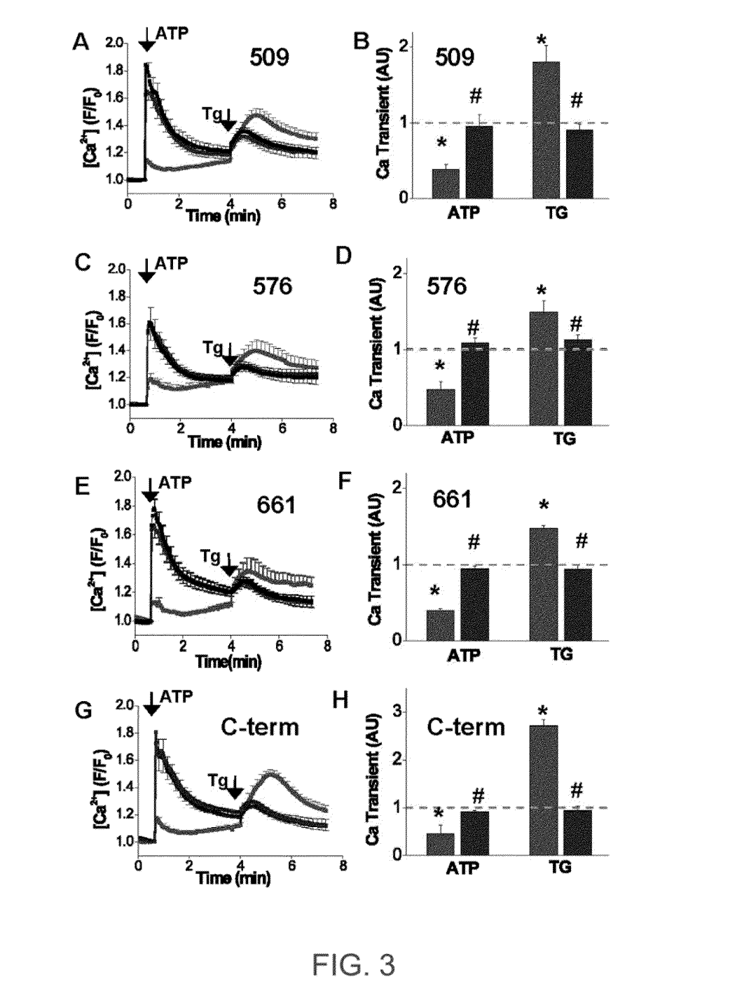

To verify that the fluorescent protein fusion tags did not disrupt SERCA catalytic function, Ca uptake was measured for all constructs using a live-cell Ca uptake assay. FIG. 3 shows that untransfected cells responded to application of extracellular ATP (arrow “ATP”) with a large cytosolic Ca transient that was detected as an increase in the fluorescence of the Ca-sensitive dye X-rhod-1 (FIG. 3, black traces). This accumulation of Ca in the cytosol was almost completely prevented in cells transfected with 2-color SERCA, suggesting that the transport activity of exogenous SERCA counterbalanced Ca release. In this experiment, heterogeneous expression levels in a population of AAV-293 cells permit comparison of transfected and untransfected cells in the same field. Notably, Tg-releasable ER Ca content was greater in cells transfected with 2-color SERCA (FIG. 3, “Tg”). Co-transfection of 2-color SERCA constructs with YFP-PLB partially restored the observed Ca transient and reduced the Tg-releasable ER Ca content, consistent with inhibition of SERCA by PLB. For all constructs, the area under the curve was quantified for ATP- and Tg-dependent Ca transients and summarized in FIGS. 3B, 3D, 3F, and 3H as normalized values relative to untransfected cells.

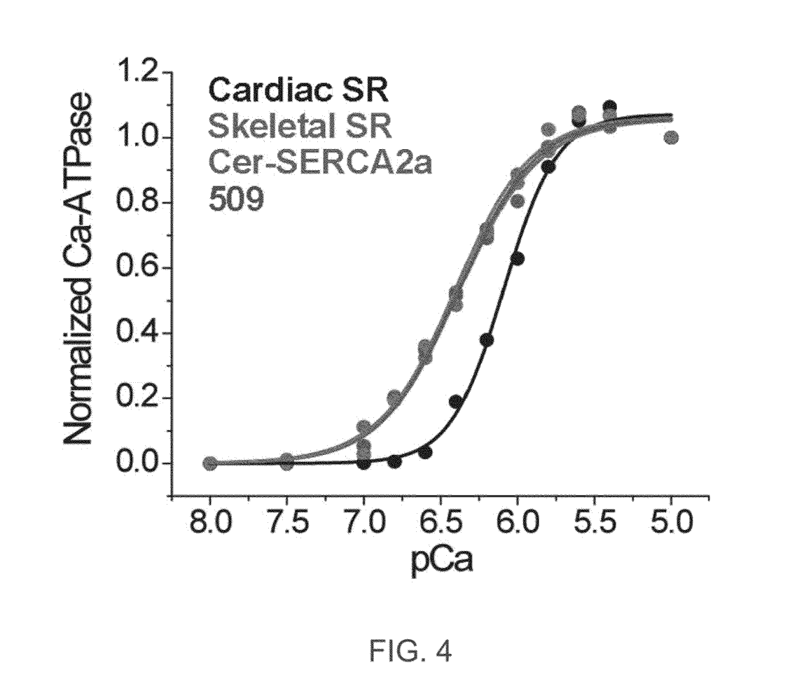

Pump activity was also evaluated for some constructs using an enzyme-linked ATPase assay performed on cell homogenates. FIG. 4 shows a comparison of the Ca-dependent ATPase activity of cardiac SR and skeletal SR controls versus homogenates of AAV-293 cells infected with adenoviruses encoding Cer-SERCA2a or 2-color SERCA construct 509 after subtraction of endogenous ATPase activity. 2-color SERCA yielded a pKCa of 6.4 and a Hill coefficient of 1.7, which is in agreement with skeletal light SR control. ATPase specific activity was determined to be 3.79±0.98 s⁻ per SERCA for Cer-SERCA2a and 4.98±1.68 s⁻¹ per SERCA for construct 509. These values compare favorably to that of porcine cardiac SR, which yielded a specific activity of 3.08±0.18 s⁻¹ per SERCA. Overall, the data indicate that the fluorescent protein fusion tags are benign for SERCA catalytic function and regulation.

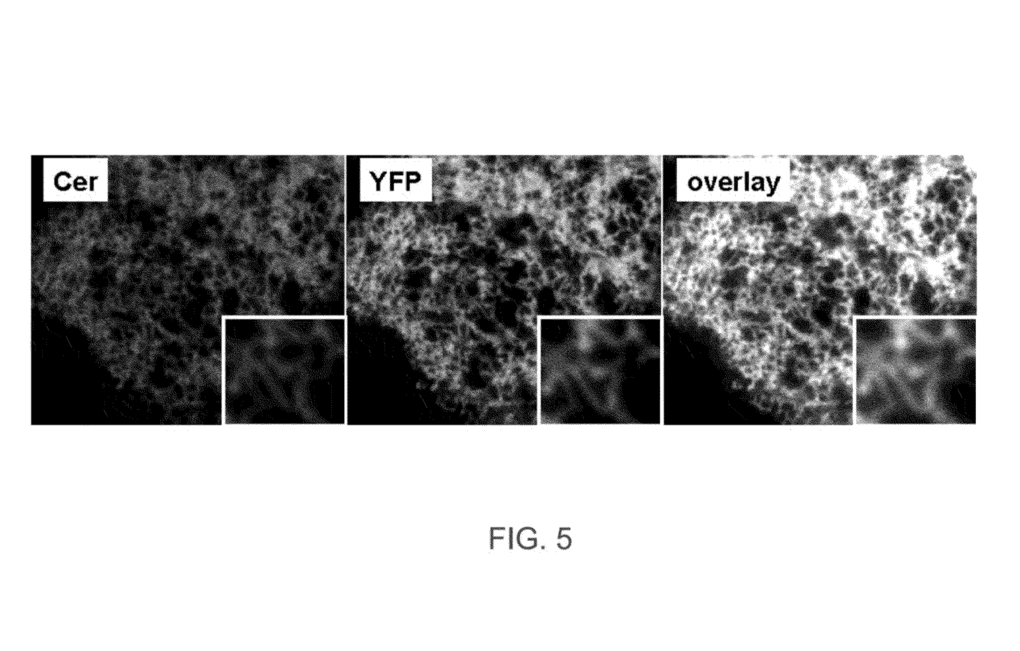

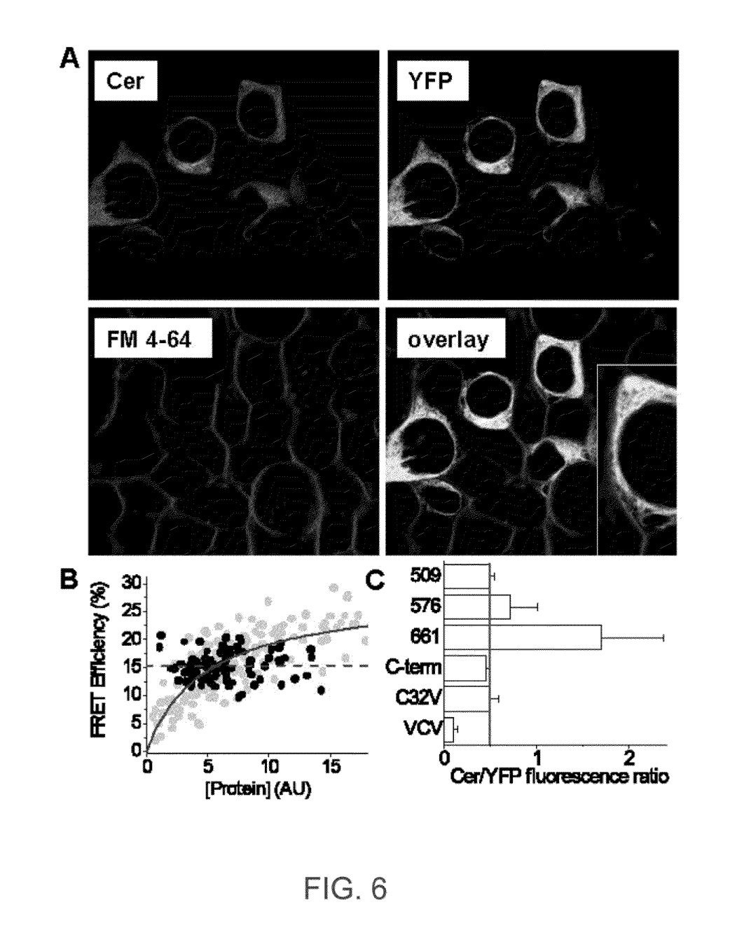

All of the constructs showed fluorescence localization patterns consistent with localization in the endoplasmic reticulum. TIRF microscopy of cells expressing construct 509 showed that Cer and YFP signals were highly colocalized in a reticulated pattern (FIG. 5). FIG. 6A shows confocal microscope images obtained by sequential acquisition of signals from Cer, YFP, and the cell-impermeant membrane dye FM 4-64. The red fluorescence of FM 4-64 was localized to the plasma membrane, while the Cer and YFP fluorescence of 2-color SERCA (509) was localized to internal, perinuclear membranes. For this construct, 0.15% FRET efficiency that was not dependent on protein expression level was observed (FIG. 6B, black points and dotted line). This concentration independence of intramolecular FRET is in contrast to previous observations of intermolecular FRET. For example, FRET from SERCA to PLB showed a hyperbolic concentration dependence (FIG. 6B, grey pots and solid line). The lack of concentration dependence suggests that the FRET observed for 2-color SERCA is not due to protein-protein interactions, such as SERCA dimerization, and that nonspecific FRET is not a significant factor for these experiments.

The observed Ca transport and ATPase activity (FIGS. 3 and 4) suggest that the sections of protein derived from SERCA were folded into the correct conformation. To test whether the donor and acceptor fusion tags were properly folded, the fluorescence of Cer and YFP were compared for all constructs. The illumination and detection configuration used in the present study have previously been determined to yield a Cer/YFP brightness ratio of 0.5. FIG. 6C shows that the control construct C32V yielded a Cer/YFP ratio of 0.5, consistent with a 1:1 stoichiometry of Cerulean and Venus fluorescent proteins. Another control composed of a Cer fused to 2 Venus yielded a much lower Cer/YFP ratio consistent with a 1:2 stoichiometry and significant quenching of the Cer protein by FRET. Two of the 2-color SERCA constructs gave the expected Cer/YFP ratio of approximately 0.5, suggesting correct maturation of the fluorescent protein tags with 1:1 stoichiometry (FIG. 6C, 509 and C-term). The other two constructs showed increased Cer/YFP ratios and a larger cell-to-cell standard deviation (FIG. 6C, 576 and 661), suggesting that the intrasequence YFP tag was not achieving uniformly correct folding or maturation. Incomplete fusion tag maturation was expected to reduce the measured FRET and decrease the dynamic response for these constructs. The apparent misfolding of 576 and 661 intrasequence YFP was not improved by reducing the cell culture incubation temperature from 37 degrees C. to 25 degrees C. (not shown).

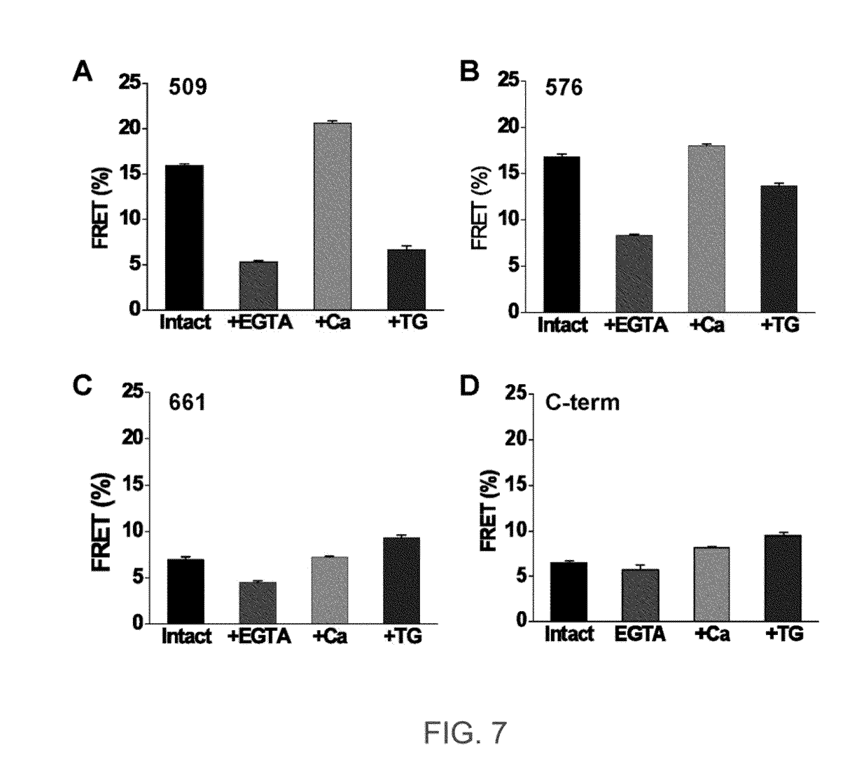

The average FRET observed for 2-color SERCA was found to depend on the YFP insertion site and the enzymatic substate of the pump (FIG. 7). For control unpermeabilized cells, the highest FRET was observed for constructs 509 (FIG. 7A) and 576 (FIG. 7B; 0.15%). These constructs have the YFP acceptor inserted in the top of the N-domain (FIG. 2B). The P-domain insertion site (661) and the C-terminal acceptor position gave the lowest initial FRET. Ionophore permeabilization in the presence of 5 mM extracellular EGTA caused a decrease in FRET for all constructs (FIG. 7) over approximately 50 minutes. Treatment with ionophore in high extracellular Ca resulted in increased FRET in several minutes. While the magnitude of the change with Ca varied with YFP insertion site (FIG. 7), the direction of the Ca-dependent FRET change was positive for all constructs, suggesting that the conformational change from E2 to E1 decreased the distance between the donor and acceptor. This is in contrast to the response of 2-color SERCA constructs to Tg, which varied in magnitude and direction depending on the YFP insertion site (FIG. 7).

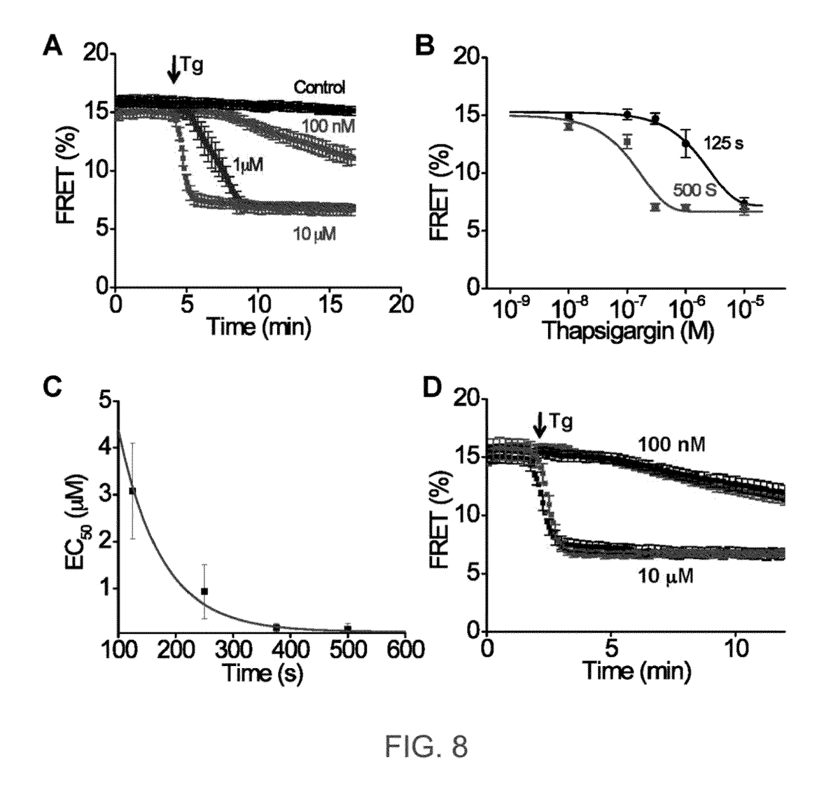

Additional characterization of construct 509 showed that the time course of the Tg-dependent FRET change was dependent on the concentration of Tg applied to the cells (FIG. 8A), with concentrations greater than 1 μM requiring tens of minutes to produce a full effect. One consequence of the slow FRET response at low concentrations of Tg is that the apparent EC₅₀ decreased over time. This is evident in FIG. 8B as a left shift in the binding curve with time. This change is quantified in FIG. 8C. The apparent upper limit estimate of EC₅₀ (obtained from the final EC₅₀ value) was approximately 100 nM, which is compatible with the subnanomolar affinity of the Tg-SERCA complex. Notably, the timecourse of Tg binding was not significantly affected by coexpression of excess PLB, regardless of Tg concentration (FIG. 8D). These data are not consistent with the proposal that PLB protects SERCA from Tg by stabilizing a conformation that is not receptive to Tg. The timecourse of the Ca dependent FRET change also showed slow kinetics.

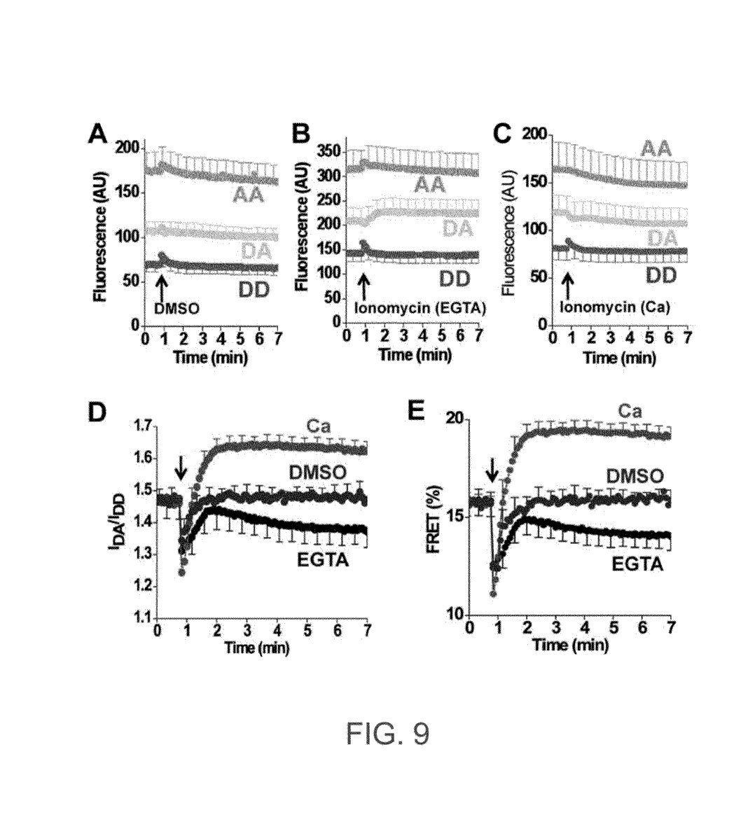

FIG. 9 shows the response of construct 509 to addition of DMSO (FIG. 9A) vehicle or ionomycin to cells in the presence of Ca (FIG. 8B) or in EGTA (FIG. 8C). The ratio of the DA/DD signals is shown in FIG. 8D, and the corresponding FRET efficiency is given in FIG. 9E. The initial negative deflection (at arrow) in FIG. 9D and FIG. 9E is an optical artifact that was transient in duration. This is believed to be attributed to the mixing that occurs with addition of a large volume of solution to the bath surrounding the cells. Note that the rate of Ca flux is limited by the slow transport of Ca by ionomycin, and this limitation is most significant for EGTA experiments in which micromolar intracellular Ca is dialyzed against nanomolar extracellular Ca. Data from experiments such as FIG. 8A or FIG. 9E are summarized in FIG. 7, using the final FRET value obtained after equilibrium was achieved.

Based on the above described tests, it is believed that SERCA intramolecular FRET is increased by Ca binding for all four 2-color SERCA constructs. This suggests a decrease in the distance between the Cer donor fluorophore and the YFP acceptor, which implies that the SERCA cytoplasmic headpiece becomes more compact after Ca binds to the SERCA transmembrane domain. The apparent donor-acceptor separation distances are summarized in Table 1. Several uncertainties apply to these values. Distances were calculated using the assumption that the average relative dipolar orientation of the probes was random (κ^{2=2/3}). Nonspecific FRET was not subtracted from the measured FRET values, because there was no evidence of a concentration-dependent increase in FRET (FIG. 6B). The position of the fluorescent chromophores is not known precisely, as they are attached with flexible 2-residue linkers to SERCA cytoplasmic domains. Finally, the FRET efficiency measured in a steady-state experiment represents an average value that may integrate the contributions of multiple structural subpopulations. All of these unknown parameters complicate comparison of absolute distance measurements with X-ray crystal structures. However, assuming that the uncontrolled factors are not significantly different for E1 and E2, the relative change in FRET was analyzed to gain insight into SERCA conformational changes.

Compared to EGTA, the apparent probe separation distance decreased with high Ca by approximately 23% (from 80 Å to 62 Å) for the most responsive construct, 509 (Table 1). This change was somewhat smaller in magnitude than the 30+Å change predicted by the first X-ray crystallographic structures (FIG. 1A). Most notably, the distance change observed here was opposite in direction compared to early crystal structure predictions. The apparent decrease in FRET distance with Ca also contrasts with previous FRET studies that used reactive dyes as donors/acceptors. This may be due to differences in labeling strategies. FRET pairs on the N- and A-domains are expected to be maximally sensitive to SERCA headpiece conformational changes, and are benign for pump function (FIG. 4). In contrast, some dye conjugation chemistries result in inactivation of SERCA catalytic activity. The Ca-dependent decrease in probe separation distance observed here suggests closure of the cytoplasmic headpiece (FIG. 1B), and this was observed for all of the 2-color SERCA constructs (Table 1). The data are compatible with recent crystallographic studies, which collectively suggest the tightest closure of the cytoplasmic headpiece is in Ca-bound (E1) substates. Specifically, if the nucleotide-free Ca₂E1 structure is excluded, a comparison of the major E1 and E2 crystal structures shows that the average separation distance between the N-terminal Cer fusion site (before residue one) and the intrasequence YFP insertion site before residue 509 decreases with Ca binding. However, the large scale of the FRET distance change was unexpected, as the nucleotide-bound states observed by X-ray crystallography all have rather closed structures (FIG. 1B), leaving little room for large amplitude translation of the A- and N-domains. It is possible that open E2 conformations have been overlooked because they are relatively disordered, without domain-domain contacts to stabilize them for crystallization.

Supporting the hypothesis of large-amplitude headpiece transitions is a recent study using fast scanning atomic force microscopy, which demonstrated 23 Å changes in the height of the SERCA headpiece relative to the surface of the bilayer during catalytic cycling. The data are also consistent with molecular dynamics simulations performed on SERCA starting at the open conformation of SERCA obtained in the first crystal structure (FIG. 1, left). In the molecular dynamics study, a large-scale conformational change was observed in which the cytoplasmic head-piece closed dramatically both in the presence and absence of Ca2+, with a slightly more closed conformation (by four Å) observed in the presence of Ca. That study showed that this extra Ca-dependent closure was likely necessary and sufficient for SERCA to reach the precise geometrical arrangement necessary for activation of ATP hydrolysis.

TABLE 1
Quantitative FRET of 2-Color SERCA Constructs
FRET Efficiency (%)

| | 510 | 577 | 662 | C-term |
|---|---|---|---|---|
| Intact cells | 15.9 ± 0.2 | 16.8 ± 0.3 | 7.0 ± 0.3 | 6.5 ± 0.2 |
| +Ca | 20.6 ± 0.3 | 18.0 ± 0.2 | 7.2 ± 0.1 | 8.1 ± 0.2 |
| +TG | 6.6 ± 0.4 | 13.7 ± 0.3 | 9.4 ± 0.2 | 9.5 ± 0.3 |
| +EGTA | 5.3 ± 0.1 | 8.3 ± 0.1 | 4.5 ± 0.2 | 5.8 ± 0.6 |

Probe Separation Distance (Å)

| | 510 | 577* | 662* | C-term |
|---|---|---|---|---|
| Intact cells | 65.7 ± 0.2 | 65.0 ± 0.2 | 76.6 ± 0.6 | 77.7 ± 0.4 |
| +Ca | 62.4 ± 0.2 | 64.1 ± 0.1 | 76.3 ± 0.2 | 74.7 ± 0.3 |
| +TG | 77.5 ± 0.8 | 67.7 ± 0.3 | 72.6 ± 0.3 | 72.5 ± 0.5 |
| +EGTA | 80.5 ± 0.3 | 74.3 ± 0.2 | 82.9 ± 0.6 | 79.3 ± 1.5 |

*incomplete fluorophore maturation for 576 and 661 may yield underestimated distance.

Of the YFP constructs described here, the least responsive was 661, which showed low initial FRET efficiency and small changes with Ca or Tg. This is consistent with inefficient maturation of the YFP tag (FIG. 6C) that is inserted in the SERCA P-domain. In the future, this may be overcome by using alternative fluorescent protein tags or different P domain labeling positions in order to study the conformational dynamics of this part of SERCA. Other variants performed better, particularly 509, which showed large changes in FRET in response to Ca and Tg. Although the fluorescent protein fusion tags are large, previous studies and present data indicate they are benign for function (FIG. 3, FIG. 4) and can report conformational changes (FIG. 7).

It was shown that steady-state FRET experiments can resolve structure changes of the Cer-SERCA-YFP constructs. The data suggest N-domain sites got farther to the N-term donor after TG, while P-domain and the C-terminus got slightly closer. For all labeling sites, FRET increased with Calcium, suggesting the pump assumes a more compact conformation with Ca-binding. Preliminary data suggest that the pump is still catalytically active, and able to pump calcium.

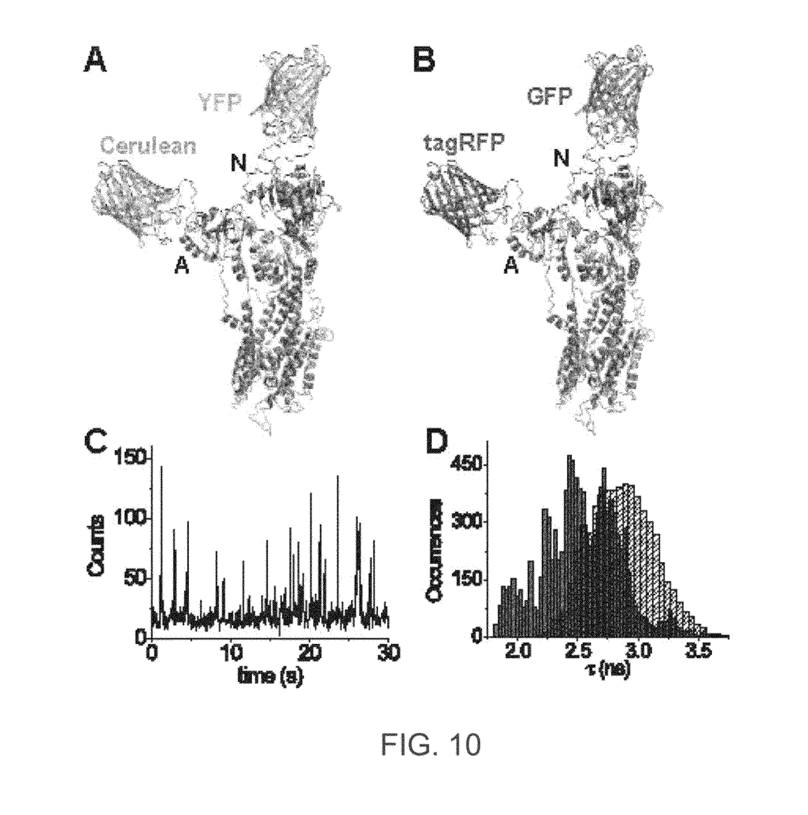

In a second set of experiments, additional testing was performed with an another 2-color SERCA, RG-SERCA, that is believed to benefit from a higher efficiency FRET pair (FIG. 10B). A quantitative spectroscopic method was also used that resolves SERCA structural heterogeneity. The goal of these additional experiments was to identify discrete structural substates of SERCA and quantify the population of those states in live cells. In particular, dynamic changes in the distribution of SERCA among several discrete conformations was measured in electrically paced adult ventricular myocytes. The RG-SERCA construct had the N terminus fused to tagRFP and EGFP was inserted as an intrasequence tag before residue 509 in the N-domain. Expression of 2-color SERCA in AAV-293 cells was performed as previously described in the first set of experiments.

In the second set of experiments, the 509 construct was again utilized as well as a RG-SERCA construct. The Cer-YFP pair had a Förster distance (R0) of 49.8 Å and the GFP-tagRFP pair had an R0 of 58.3 Å. Expression of 2-color SERCA in AAV-293 cells was performed as previously described in reference to the first set of experiments. Briefly, cells were transfected with plasmids encoding 2-color SERCA using the MBS mammalian transfection kit (Stratagene). The transfected cells were trypsinized and re-plated onto poly-D-lysinecoated glass bottom dishes and allowed to attach for 2-4 hours before being used for measurements. Cotranfection of 2-color SERCA with non-fluorescent S16A-PLB or S16E-PLB was performed at a plasmid molar ratio of 1:10. For single molecule experiments, the protein was solubilized in detergent as follows: 0.1% dodecylphosphocholine (DPC) (Sigma Aldrich) in phosphate buffered saline was gently layered over AAV-293 cells expressing 2-color SERCA, and incubated at room temperature for 45 minutes. The solution was withdrawn and centrifuged at 16,000 g for 10 min, and the supernatant was transferred to Matek chambered coverglass for spectroscopy.

Adenoviral vectors of 2-color SERCA were prepared using AdEasy system (Stratgene, La Jolla, Calif.). Adult cardiac ventricular myocytes were enzymatically isolated from adult New Zealand White rabbits. Myocytes were transferred to culture vessels and washed with fresh PC-1 medium (Lonza, Basel, Switzerland). 2-color SERCA adenoviruses were added at a multiplicity of infection of 1000. Cells were paced for 48 hours in culture using a C-Pace EP pacer (IonOptix, Milton, Mass.) set to 10 volts with a frequency of 0.1 Hz and 5-ms pulse duration. During spectroscopy experiments, electrical pacing of cardiac myocytes was performed with a stimulator (Grass S44, Astro-Med, Inc., West Warwick, R.I.), with 50 V stimulation, 5 ms duration, 0.25 Hz.

Time correlated single-photon counting (TCSPC) and fluorescence imaging were performed on an inverted confocal microscope (TCS-SP5, Leica Microsystems) equipped with a 63×1.20 NA water immersion objective (Leica Microsystems) and a pulsed Ti-Sapphire laser (Coherent Inc.), with excitation at 840 nm. Emission was split by a dichroic filter centered at 560 nm and passed through filters of 500-550 nm (for EGFP fluorescence) and 607-683 nm (for Tag-RFP fluorescence). Avalanche photo diodes (SPCM-AQRH, Perkin Elmer) were used for detection of photons. The signals from the photo diodes passed to a pulse inverter (APPI-D, Becker & Hickl GmbH) and 20-dB attenuator and a TCSPC router (HRT-41, Becker & Hickl GmbH) for simultaneous data collection of both emission channels. The signal output and the routing information from the router were transmitted to a TCSPC card (SPC-830, Becker & Hickl GmbH) to record temporal information for each detected photon. A synchronization reference signal for the TCSPC measurements was obtained by directing a portion of the excitation laser onto a photo diode (PHD-400-N, Becker & Hickl GmbH). Temporal information was recorded as two different time tags for each detected photon: t₁, the microscopic arrival time relative to the previously measured synchronization reference signal, and t₂ indicating the macroscopic arrival time of the photon measured with respect to the start of the experiment. Time tag t₁ provides information about ns scale fluorescence lifetime and t₂ measures the timecourse of the experiment with ms time resolution. TCSPC data were analyzed with a sliding-scale method to obtain fluorescence lifetime (τ) histograms from recorded photon arrival times. Briefly, the train of photons detected in the FRET donor channel was analyzed in overlapping blocks of 200 consecutive photons, with τ determined for each block by maximum likelihood estimation (MLE). A histogram of the t₁ arrival times of the photons was used to calculate the likelihood function. The probability distribution for this histogram was approximated by a single exponential decay of the form e^{-t/τ}. Maximizing the logarithm of the likelihood function for the single exponential decay yields

$$\frac{ω}{1 - {e}^{- ω / τ}} - \frac{T}{{e}^{T / τ} - 1} = \frac{{\sum }_{i} {n}_{i} {t}_{i}}{N} ,$$

where nᵢ indicate the number of photons in the i^{th} bin of the histogram and

$$N = {\sum }_{i} {n}_{i} .$$

ω is the width of the time bins in the histogram and tᵢ=iω with 0≦tᵢ≦T. The root of this equation yields the MLE estimate of the τ value of the fluorophore for the corresponding t₂ time bracket. The analysis was automated with a custom MatLab program. The method was validated by determining the fluorescence lifetime of Rhodamine 6G in water at room temperature. FRET efficiency was calculated from measured τ values according to the relationship (1−(τ_{DA}/τ_{D})), where τ_{DA} values are measurements of 2-color SERCA fluorescence lifetime and τ_{D} is the average τ of the donor-alone control. FRET efficiency histograms were fit to a sum of four Gaussian peaks with Origin (OriginLab Corp., USA). All fit parameters (the center value, the width and area of each Gaussian) were iteratively varied until convergence was obtained. Parameter mean values were obtained from five independent measurements.

The fluorescent protein FRET pairs for Cer-SERCA-YFP and RG-SERCA have different R₀, τ_{D}, and emission spectra. The constructs also have reciprocal donor/acceptor positions. Despite these differences, the distances calculated from FRET substates were very similar for Cer-SERCA-YFP and RG-SERCA (Table 3). The agreement of these values suggests that the probe separation distance estimate was not dominated by κ² or beat-to-beat changes in pH or autofluorescence.

FRET efficiency trajectories calculated for each single molecule transit were obtained for analysis of SERCA structural dynamics. FRET efficiency values measured from overlapping blocks of 200 consecutive photons were assigned to specific conformational States I-IV identified from previous Gaussian fitting of FRET efficiency histograms (Supplemental Table 1). The full width at half maximum of each Gaussian distribution was used as a threshold for substate assignment. The duration of time spent in each FRET state before transition to another FRET state was quantified, and a histogram of observed dwell times was generated using a custom analysis routine in MATLAB. The histogram of the dwell times was fit by a biexponential function.

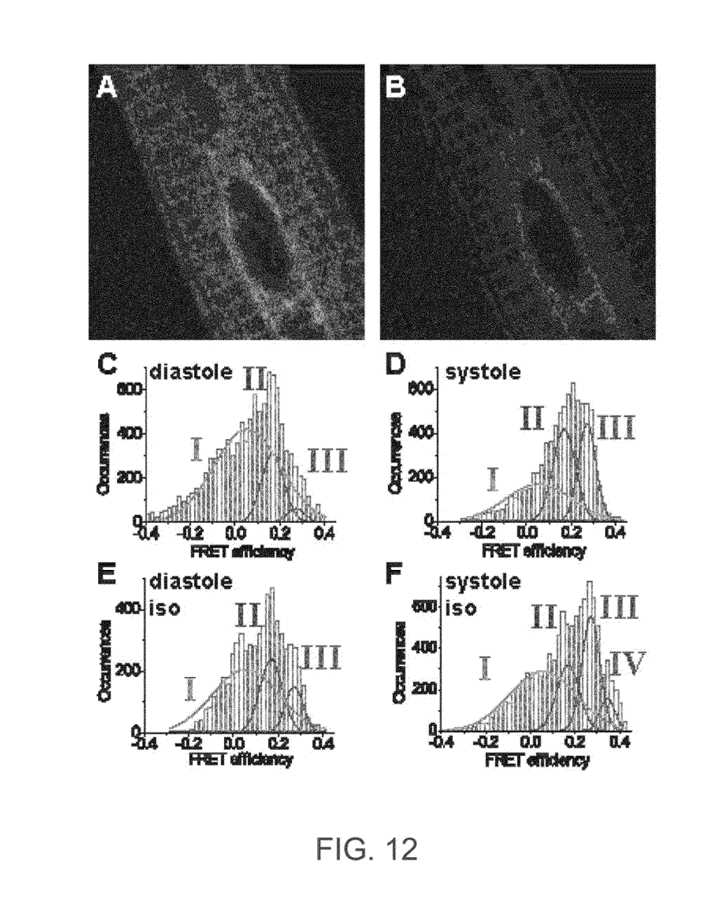

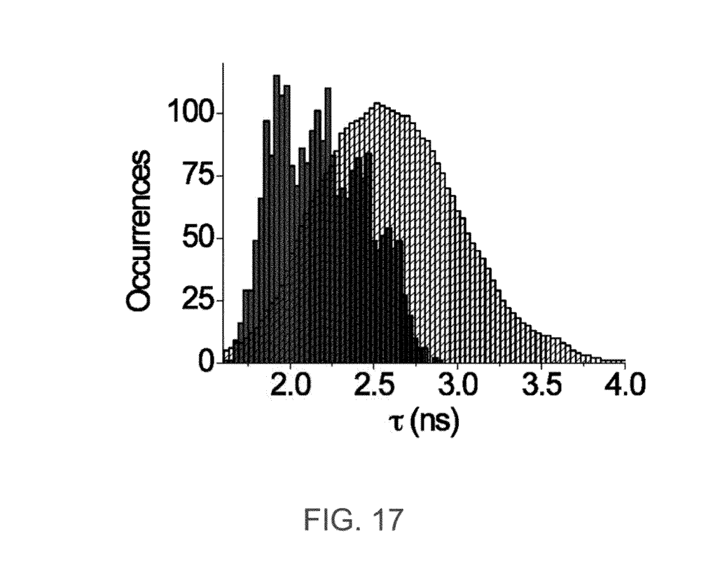

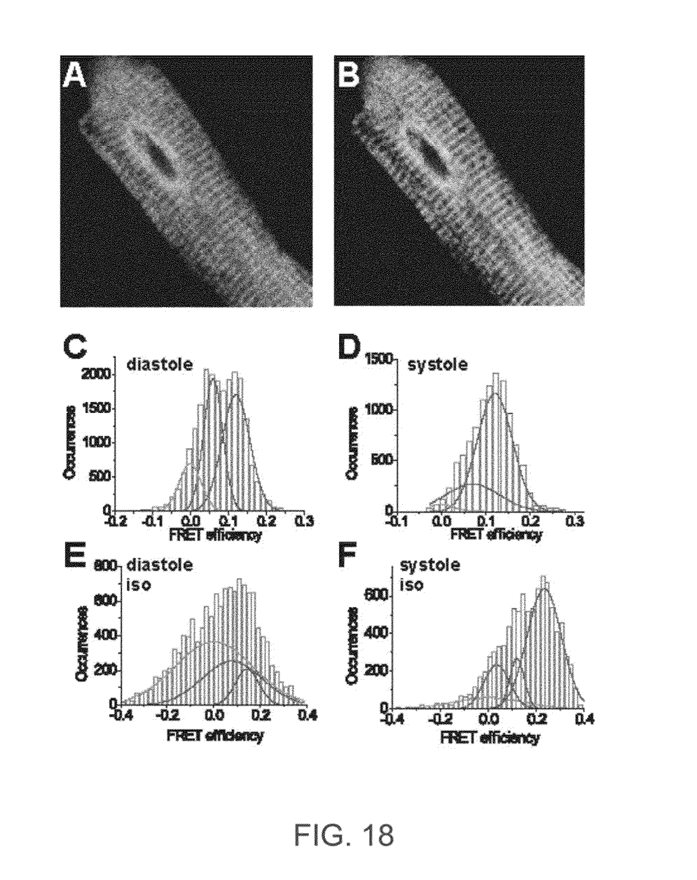

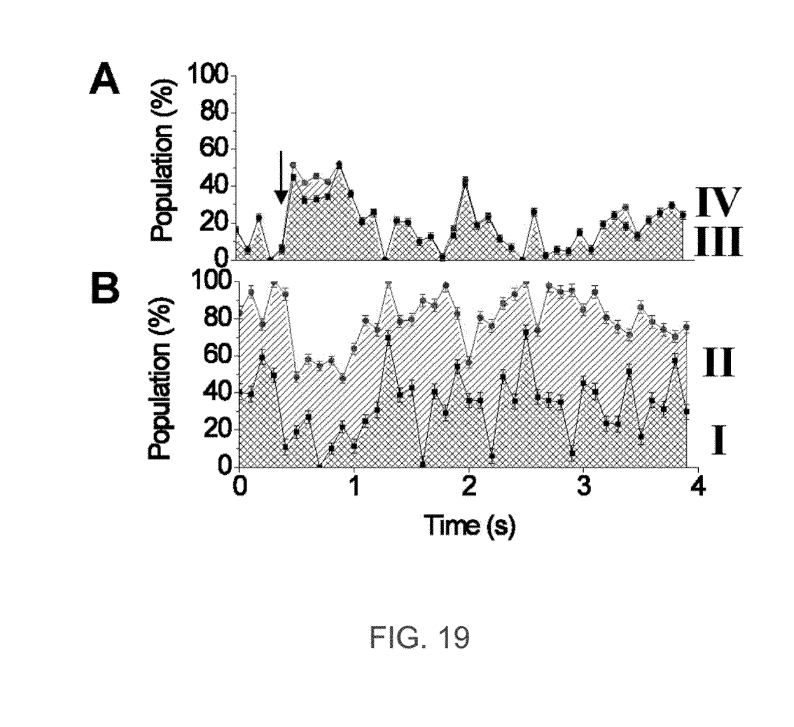

Single molecule FLDA experiments were performed with Cer-SERCA-YFP. A histogram of measured τ values revealed a large range of lifetimes from 1.2 ns to 2.9 ns (FIG. 17). The average lifetime was decreased compared to a non-FRET control (donor only) Cer-SERCA (FIG. 17, black striped bars). The CY-SERCA τ histogram showed four distinct maxima, as was also observed for RG-SERCA (FIG. 10D). Cer-SERCA-YFP expressed in cardiac myocytes showed a striated localization pattern (Supplemental FIGS. 18A and 18B) similar to that observed for RG-SERCA (FIGS. 12A and 12B). Three distinct conformations of Cer-SERCA-YFP are suggested from the FRET histogram, with a high FRET state increased during systole (FIG. 18D) at the expense of lower FRET states. A very high FRET state was observed in systole after iso stimulation. To quantify time-dependent changes in SERCA substate populations during the course of the contraction/relaxation cycle, fluorescence lifetime data were acquired during 16 consecutive contractions of a myocyte paced at 0.25 Hz. FLDA data were gated from the four second pacing cycle, combining data from all cycles into 40 summary FRET histograms (100 msec time resolution). These histograms were globally fit with a four peak Gaussian function, where peak positions and widths were fixed according to values previously determined above and the peak areas were independent. Calculated peak areas are provided as the percent of total histogram area in FIG. 19.

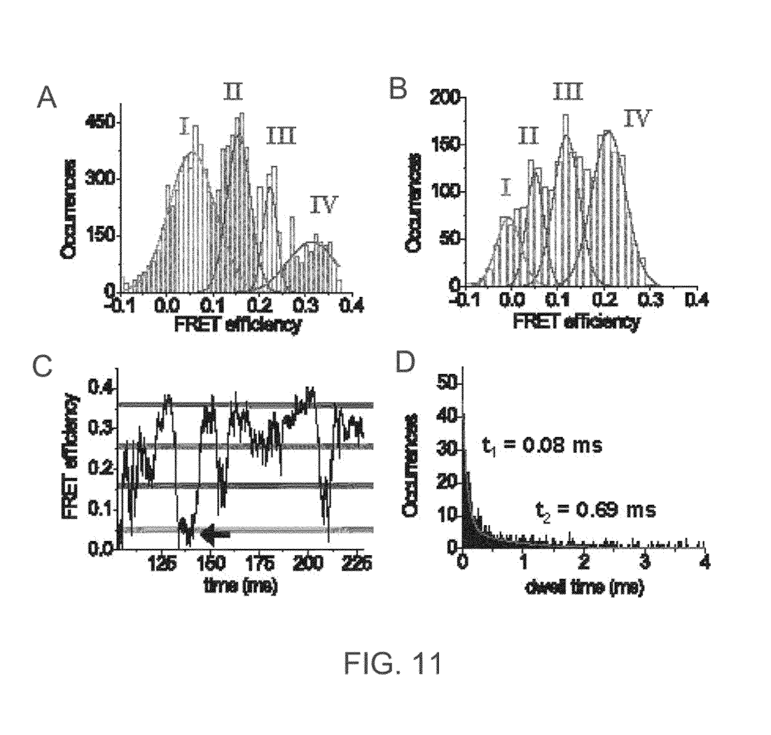

To survey resolvable conformations of SERCA in vitro, pulsed excitation single molecule fluorescence spectroscopy was performed with fluorescence lifetime distribution analysis (FLDA) using RG-SERCA solubilized in dodecylphosphocholine (DPC). FIG. 10C shows the GFP donor fluorescence emission of RG-SERCA, with bursts of photons indicating the transit through the excitation volume of detergent micelles containing individual 2-color SERCA molecules. Fluorescence excited-state lifetimes (τ) were quantified using a sliding scale analysis method. A histogram of measured τ values revealed a large range of lifetimes from 1.8 ns to 3.3 ns (FIG. 10D). The average lifetime was decreased compared to a non-FRET control (donor only) GFP-SERCA (FIG. 10D, black striped bars), consistent with robust intramolecular FRET for RG-SERCA. Moreover, in contrast to the donor-alone τ distribution, the RG-SERCA histogram exhibited four distinct maxima (FIG. 10D). The resolved τ subpopulations suggest at least four discrete FRET states as the proteins diffuse through the excitation volume, consistent with at least four discrete conformations of the pump. Similar results were obtained for Cer-SERCA-YFP. FRET efficiency histograms for RG-SERCA (FIG. 11A) or Cer-SERCA-YFP (FIG. 11B) were calculated from measured τ values according to the relationship E=(1−(τ_{DA}/τ_{D})), where τ_{DA} values are measurements of 2-color SERCA fluorescence lifetime and τ_{D} is the average τ of the donor-alone control. The measured donor lifetime for Cer-SERCA control was 2.6 ns, slightly less than the previously published Cer τ of 2.99 ns. Fusion of Cer to a target protein has been shown to decrease τ. A τ of 2.86 ns was obtained for GFP (FIG. 10D), which was similar to previously published values of 2.96 ns. A four peak Gaussian fit resolved FRET subpopulations with center values of 0.05, 0.16, 0.25 and 0.34 for RG-SERCA (FIG. 11A) and −0.01, 0.07, 0.12, 0.21 for Cer-SERCA-YFP (FIG. 11B). These discrete states are denoted as I, II, III, and IV, respectively. The increased FRET efficiency values observed for RG-SERCA substates compared to Cer-SERCA-YFP are consistent with a longer Förster distance (R₀) for the GFP-tagRFP pair (58.3 Å) vs. the Cer-YFP pair (49.8 Å). The apparent separation distances of the fluorescent protein chromophores were calculated according to the Förster relationship. For Cer-SERCA-YFP, probe separation distances for states II, III, and IV were 78, 69, and 62 Å. The FRET efficiency for State I was too low to determine a probe separation distance for this FRET pair. For RG-SERCA, the separation distance for states I, II, III, and IV were 94, 77, 70, and 65 Å, respectively, in good agreement with the measurements made with the Cer-YFP pair.

Detergent-solubilized single molecules yielded time-trajectories of up to 800 ms in duration. These showed apparent stochastic transitions between high FRET and low FRET conformations (FIG. 11C). For comparison, the FRET efficiency of states I-IV identified from distribution analysis (FIG. 11A) are marked in (FIG. 11C) as horizontal lines. The pump was observed to transiently sample the open conformation (State I) before returning to high FRET states during the time trajectories (FIG. 11C, arrow). Such reversibility suggests that the low FRET state does not arise from denatured or proteolyzed 2-color SERCA. Analysis of the kinetics of single molecule structure transitions revealed that SERCA samples states I-IV for variable durations. A histogram of measured substate persistence revealed a biphasic distribution of SERCA dwell times (FIG. 11D). An exponential fit of this distribution showed the transitions fall into two distinct time regimes. The fast structure fluctuations (80 μs) may represent Brownian dynamics of the N- and A-domains while the slower fluctuations (690 μs) may correspond to catalytic transitions between discrete enzymatic substates. The overall rate of transitions increased from 99±55 to 200±61 transitions per minute with the addition of 2 μM Ca.

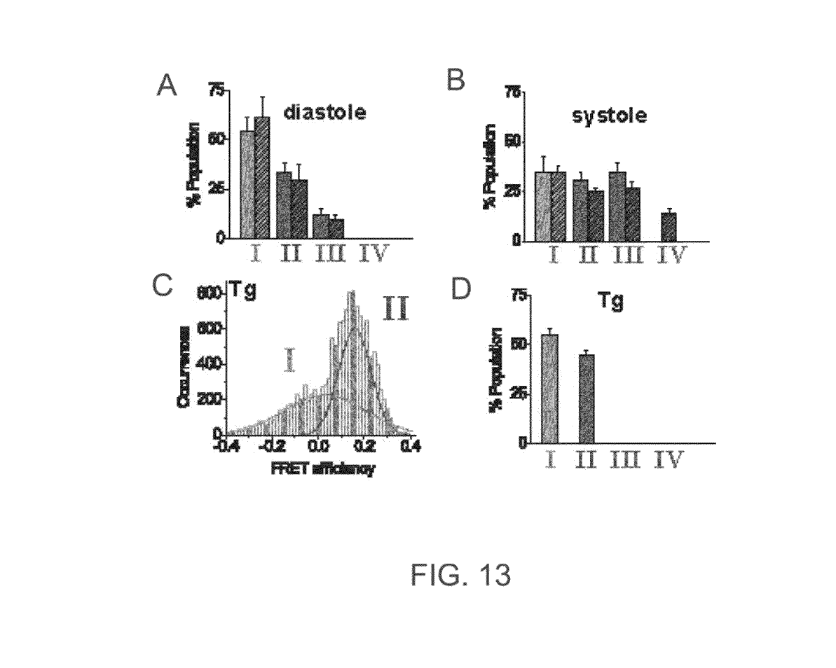

To investigate the structural heterogeneity of SERCA in cardiac muscle, 2-color SERCA was expressed in cultured enzymatically isolated rabbit myocytes. FIGS. 12A and 12B show confocal fluorescence images of RG-SERCA at 40 hours post-infection. 2-color SERCA fluorescence was distributed in striations and longitudinal streaks, suggesting localization in the junctional and transverse sarcoplasmic reticulum, respectively. The localization of RG-SERCA and Cer-SERCA-YFP were similar to mCer-SERCA2a localization in adult cardiac myocytes. Cardiac myocytes expressing a low concentration of RG-SERCA were selected for FLDA. Ca release and contraction/relaxation were induced by electrical pacing. Since τ is independent of fluorescence intensity, FLDA measurements were not subject to motion artifacts generated by myocyte contractions. FIGS. 12C and 12D show examples of FRET efficiency histograms obtained during diastole (relaxation) and systole (contraction), respectively. The distributions were well described by a fit to three Gaussian subpopulations, consistent with three discrete FRET states, corresponding to at least three distinct contemporaneous conformations of SERCA. The parameters of the Gaussian fits were all freely variable, so it is noteworthy that the center FRET efficiency values of the subpopulations closely matched states I, II and III previously identified in detergent solubilized SERCA (as in FIGS. 10 and 11). Interestingly, the high FRET state (IV) observed in single molecule experiments (FIGS. 11A and 11B) was not detected under these conditions (FIGS. 12C and 12D). The relative population of each state was quantified from the area under the Gaussian for each independent experiment. Combined data are summarized in FIGS. 13A and 13B as mean±SE. Overall, the majority of SERCA resided in low FRET states (I and II) during diastole. With the increase in Ca during systole the population of high FRET State III increased from 12.3% to 34.7% of total. Gains in State III came at the expense of the population of low RET efficiency State I and to a lesser extent, State II (FIGS. 13A and 13B). The data suggest that Ca binding to SERCA stabilizes a compact, high FRET structure (III) (FIG. 12D), consistent with the previous observation that 2-color SERCA average FRET increased with Ca.

The effect of adrenergic stimulation on SERCA structural heterogeneity was also investigated. The effect of 8 agonist (isoproterenol) was not apparent during diastole, with the majority of SERCA assuming low FRET conformations (states I and II) (FIG. 12E), similar to untreated myocytes (FIG. 12C). However, the effect of 8 agonist was revealed during systole, with a significant fraction of pumps switching to the latent high FRET state (IV) (FIG. 12F) that was first observed in detergent-solubilized SERCA (FIGS. 12E and 12F). The observed high FRET efficiency indicates this unique structure is characterized by a very tightly closed cytoplasmic headpiece. One possible interpretation of this result is that the high FRET state results from unbinding of PLB from SERCA. An alternative interpretation is that PLB remains associated with SERCA after phosphorylation, and together with elevated Ca, stabilizes SERCA in a new conformation. These two alternative mechanisms are directly tested below. It is interesting that a relatively small fraction of the SERCA population in cultured myocytes switch from the prevailing low FRET states in diastole (FIGS. 12C and 12E) to high FRET states in systole (FIGS. 12D and 12F). Indeed, under physiological conditions the population of SERCA is not expected to synchronously transition between various substates en masse. The density of SERCA in the SR is such that each pump would only have to turnover once to completely sequester available Ca. The results underscore the value of lifetime distribution analysis for handling protein population heterogeneity. Conventional FRET measurements report the average of all contemporaneous conformations, blurring substates and reducing the apparent magnitude of conformational changes.

Overall, experiments with electrically paced cardiac myocytes suggest that low FRET states (I, II) correspond to Ca-free (E2) conformations while high FRET states (III, IV) correspond to Ca-bound conformations. Consistent with this interpretation, Tg treatment abolished states III and IV, leaving SERCA approximately evenly divided between states I and II (FIGS. 13C and 13D). Tg inhibits SERCA activity, and is commonly used to lock SERCA into a state that is taken as a surrogate for E2. Others have demonstrated that SERCA cytoplasmic domains are still dynamic after Tg binding; the present data further suggest that Tg-bound SERCA in the E2 enzymatic substate samples at least two discrete conformations.

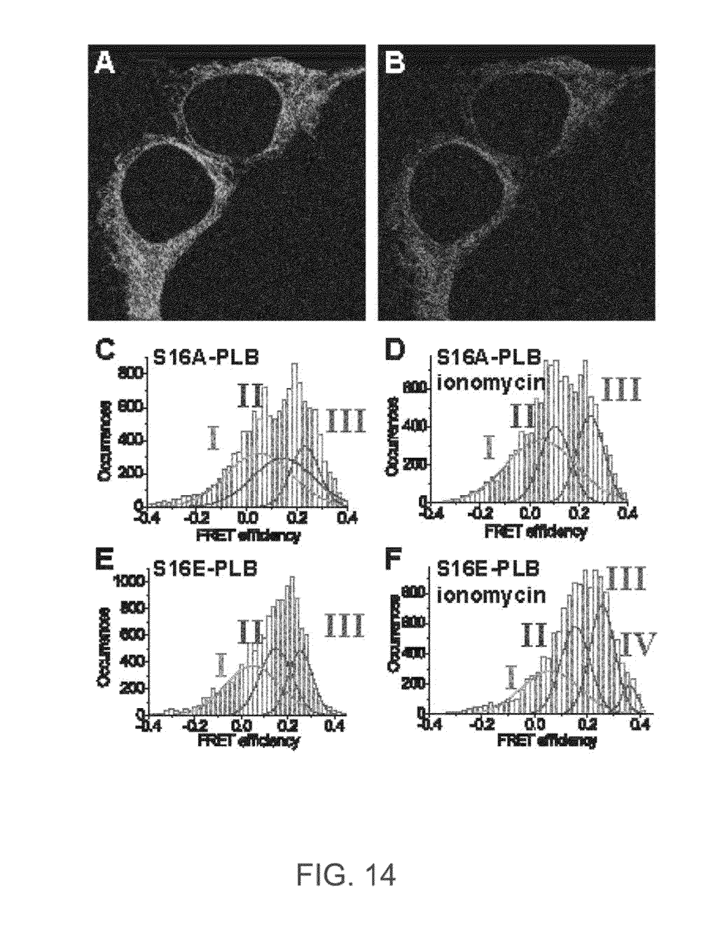

To directly test whether State IV represents a PLB-free SERCA structure or a unique phosphorylated PLBSERCA regulatory complex conformation, the distribution of RG-SERCA conformations was quantified in AAV-293 cells, which lack endogenous PLB. Confocal microscopy revealed the expected co-localization of the fluorescence of the GFP (FIG. 14A) and tagRFP (FIG. 14B) in internal membrane structures, as was observed previously for Cer-SERCA-YFP, Cer-SERCA and CFP SERCA. The FRET efficiency histogram did not present the high FRET State IV, whether SERCA was expressed alone (Table 2) or with non-phosphorylatable mutant S16A-PLB (FIGS. 14C and 14D). Interestingly, coexpression of PLB containing a mutation that mimics phosphorylation by PKA (S16E-PLB) restored the population of state (IV), but only in cells that were treated with ionomycin to increase cytosolic Ca (FIG. 14F). It is not clear why the 4th state, clearly resolved in detergent solution (FIGS. 11A and 11B), is suppressed in heterologous cells (FIGS. 14C-E) and cardiac myocytes (FIG. 12C-E). It is also not known why the combination of Ca and PLB phosphorylation uncovers this latent conformation (FIG. 12F, FIG. 14F). The data are not consistent with the model in which phosphorylation and Ca relieve SERCA inhibition by dissociating the PLB-SERCA regulatory complex, since State IV is not observed in the absence of PLB. Previously, evidence has been provided that relief of SERCA inhibition does not require dissociation of PLB. It was believed that PLB phosphorylation alters the quaternary conformation of the PLB-SERCA complex, without dissociation of the proteins. State IV observed here may correspond to this dis-inhibited conformation, a unique structure with a very tight headpiece conformation (probe separation distance 62-65 Å). This state has not yet been observed by X-ray crystallography, as diffraction quality co-crystals of PLB and SERCA have not been obtained. Some reports suggest that phosphorylated PLB provides SERCA with an alternative kinetic pathway, enhancing its catalytic efficiency. The high FRET State IV observed here may represent an activated intermediate substate in this putative alternative pathway.

TABLE 2
Changes in the relative population of discrete FRET substates in AAV-293
cells, expressed as percent of total. Values are mean ± SE.

| | S16-PLB | | S16E-PLB | | 10 μM | no |
|---|---|---|---|---|---|---|
| State | Low Ca | High Ca | Low Ca | High Ca | Tg | PLB |
| I | 47.2 ± 4.8 | 46.8 ± 3.7 | 41.5 ± 4.4 | 33.6 ± 2.3 | 62.0 ± | 12.8 ± |
| | | | | | 7.2 | 3.7 |
| II | 34.1 ± 5.4 | 35.6 ± 5.5 | 38.3 ± 4.9 | 30.6 ± 5.3 | 38.0 ± | 58.2 ± |
| | | | | | 7.2 | 12.5 |
| III | 18.6 ± 4.1 | 17.6 ± 2.5 | 20.2 ± 3.6 | 29.4 ± 5.0 | — | 28.2 ± |
| | | | | | | 12.0 |
| IV | — | — | — | 6.3 ± 1.5 | — | — |

It is noteworthy that there is a 30 Å difference in distance between the most extreme open E2 (I) and closed E1 states (IV) (Table 3). This is a larger difference than would be predicted from X-ray crystal structures. Significant structures from recent X-ray crystallography studies show on average a of about six A difference in the distance between the FRET donor and acceptor fusion sites for E1 structures versus E2 structures. A significant outlier is the first E1 crystal structure 1SU4, characterized by a widely open headpiece structure. It has been proposed that this was an artifact arising from the absence of nucleotide in the crystallization conditions. While we did not directly detect an open E1 structure, the presence of a very low FRET E2 state with a 94 Å probe separation distance suggests that the 1SU4 structure represents a real conformation that is significantly populated in vivo (Table 4). It is believed that it is unlikely that the low FRET state arises from partially expressed proteins lacking the acceptor fluorophore, as the pump was observed to reversibly sample this state (FIG. 11C). Moreover, State I was observed for both RG-SERCA (FIG. 11A) and Cer-SERCA-YFP (FIG. 11B), which have a different order of translation of donor and acceptor probes.

TABLE 3
The FRET efficiency of discrete FRET substates and corresponding
fluorescent protein chromophore separation distances, obtained
from isostimulated cardiac myocytes. Distances were calculated
using R0 values of 49.8 Å for CY-SERCA and 58.3 Å
for the RG-SERCA. Values are mean ± SE. (ND = not determined)

| | FRET Efficiency | | Probe Separation Distance (Å) | |
|---|---|---|---|---|
| | Cer-SERCA- | | Cer-SERCA- | |
| State | YFP | RG-SERCA | YFP | RG-SERCA |
| I | −0.004 ± 0.004 | 0.053 ± 0.002 | (ND) | 94.2 ± 0.5 |
| II | 0.065 ± 0.004 | 0.154 ± 0.002 | 77.8 ± 0.9 | 77.4 ± 0.2 |
| III | 0.120 ± 0.006 | 0.253 ± 0.002 | 69.4 ± 0.7 | 69.8 ± 0.1 |
| IV | 0.210 ± 0.006 | 0.349 ± 0.003 | 62.1 ± 0.4 | 64.7 ± 0.2 |

TABLE 4
Changes in the relative population of discrete FRET
substates in cardiac myocytes. Values are mean ± SE.

| | Control | | 100 μM | | 10 μM |
|---|---|---|---|---|---|
| State | Diastole | Systole | Iso Diastole | Systole | Tg |
| I | 54.5 ± 6.8 | 34.9 ± 7.6 | 61.3 ± 10.7 | 34.9 ± 2.8 | 55.5 ± 3.0 |
| II | 33.2 ± 5.3 | 30.4 ± 4.5 | 29.2 ± 8.4 | 24.9 ± 2.0 | 44.5 ± 3.0 |
| III | 12.3 ± 3.1 | 34.7 ± 5.0 | 9.4 ± 2.6 | 26.4 ± 3.5 | — |
| IV | — | — | 0.1 ± 0.1 | 13.8 ± 2.8 | — |

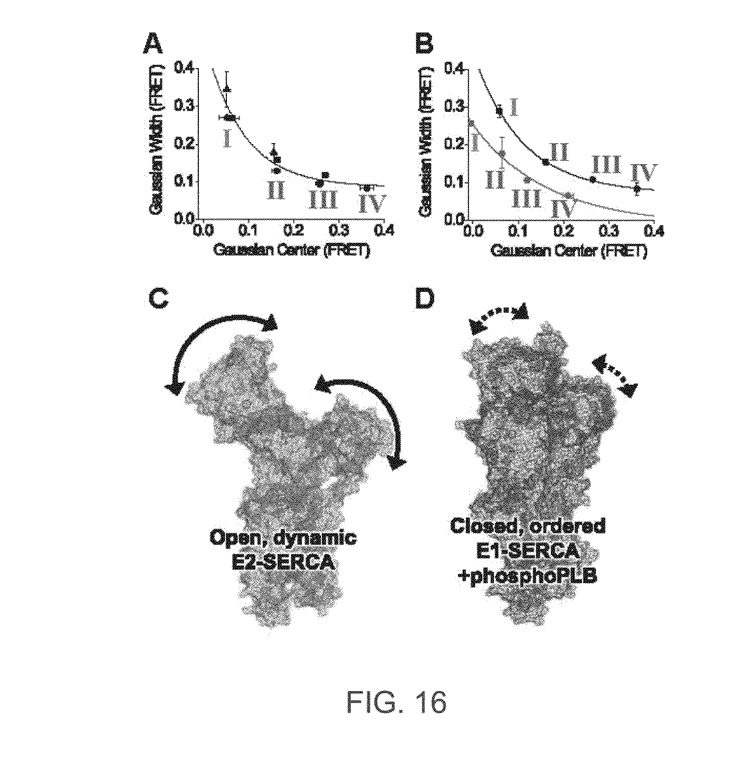

In addition to revealing the intrinsic FRET efficiency of discrete FRET states, Gaussian fitting also provided estimates of the variability of FRET observed for each state. These parameters are obtained from the peak center position and peak width, respectively. In general, histograms obtained from dilute single molecules in detergent solution were better resolved than those obtained from a higher density of fluorescent molecules in cell membranes. This is consistent with the expected ensemble averaging of multiple molecules diffusing simultaneously through the excitation volume in the cell. However, despite some loss of peak resolution in live cell experiments, it was still possible to observe an inverse relationship between Gaussian width and peak center position (FIG. 16A), irrespective of iso or Tg treatment. The data show that as FRET increases, the variability of FRET decreases. Cer-SERCA-YFP showed the same trend, and the peak widths were similar to those obtained for RG-SERCA, though the reduced Cer-YFP R₀ resulted in correspondingly decreased FRET for each State (FIG. 16B). That the two constructs gave similar peak widths despite different Förster distances supports the conclusion that the Gaussian widths are set by SERCA structural disorder. Specifically, the inverse relationship of peak width and peak center position indicate that the domain separation distance becomes increasingly well defined with closure of the cytoplasmic headpiece. Overall, the results suggest a high degree of cytoplasmic domain structural disorder for open structures, and a well-defined, rigid architecture for compact structures. It is possible that high-FRET, tightly closed conformations are stabilized by domain-domain contacts, while open conformations are unconstrained and floppy. The functional consequence of dynamic open architecture may be enhanced mobility of the nucleotide binding domain during its Brownian search for ATP substrate. It has been proposed that Ca-free (E2) SERCA can assume an open headpiece conformation. This hypothesis is compatible with the present data. Specifically, several dynamic, low FRET structures were observed in low Ca (I, II), and these structures are stabilized by Tg. It is possible that a putative “open E2” conformation, a dynamically disordered conformation, is unsuitable for crystallization. Conversely, the compact and ordered structure of State IV (E1-SERCA bound to phosphorylated PLB) make it an interesting candidate for high-resolution structure studies. Previously co-crystallization studies have focused on capturing the regulatory complex of PLB with E2-SERCA.

In view of the above, it is believed that the new FRET constructs were properly localized, functional, and responsive to conformational changes. Reversible transitions between four discrete conformations are consistent with fast (80 μs) Brownian motions and slow (690 μs) catalytic motions. These are large-amplitude transitions, producing an about 30 Å change in FRET pair separation distance. Low FRET states are consistent with open, dynamic structures, and prevail in the low Ca conditions that favor the SERCA E2 enzymatic substate. High Ca, which favors SERCA E1 substate, stabilizes high FRET states, with closed, rigidly ordered cytoplasmic headpiece conformations. The data are consistent with previous conclusion that the SERCA cytoplasmic headpiece closes with Ca-binding. Significantly, phosphorylation of phospholamban does not dissociate it from SERCA, nor does Ca-binding to SERCA abolish the regulatory complex. Instead, Ca-binding to SERCA and phospholamban phosphorylation together are believed to induce the SERCA cytoplasmic headpiece to sample a unique high FRET conformation. Taken together, the results predict several novel states that are not represented in the available X-ray crystal structures: an E2 state with a dynamically disordered open cytoplasmic headpiece (FIG. 16C), and a tightly closed E1-Ca-SERCA bound to phosphorylated PLB (FIG. 16D).

The constructs are distinct from previous approaches in that they use only genetically encoded fluorophores. Thus, they do not require labeling with exogenous dyes. The plasmid DNA encoding the constructs are suitable for transfection into mammalian cells. The encoding DNA can be transfected into mammalian cells by standard methods, or stable cell lines can be created, making the supply of drug screening cells indefinitely expandable. The ligand-response of the constructs is large and easily resolved by steady-state fluorescence measurements of live cells. All of the constructs are adaptations of the SERCA protein thereby retaining SERCA's enzymatic functions and ability to bind the two major ligands tested above; calcium and the drug thapsigargin. Experiments show that the pumps are still catalytically functional. Three of the constructs exploit intrasequence labeling of the N- and P-domains.

Potential applications of the constructs include reporting of the structural state of the enzyme during normal function. Unlike prior methods, the engineered proteins can sense changes in the conformation of SERCA itself, rather than measuring changes in the interaction of SERCA and another protein. The principal intended use of these constructs was to perform fluorescence correlation spectroscopy experiments to observe rates of structure transitions under various physiological conditions. In addition, the constructs may be used as targeted calcium sensors. The probes may be useful for measuring localized release of calcium from the sarco(endo)plasmic reticulum with high spatial resolution. Further, the constructs may be used as fluorescent sensors for high-throughput screening of drug libraries.

While the invention has been described in terms of specific embodiments, it is apparent that other forms could be adopted by one skilled in the art. For example, the physical location of the intrasequence tag could differ from that shown, and materials and processes other than those noted could be used. Therefore, the scope of the invention is to be limited only by the following claims.

1. An engineered protein for calcium handling within human cells, the engineered protein having a two-color SERCA construct comprising three component proteins fused together, the three component proteins comprising a blue fluorescent protein (cerulean donor), SERCA2a and a yellow fluorescent protein (YFP acceptor), or a red fluorescent protein (tagRFP acceptor), SERCA and a green fluorescent protein (GFP donor).

2. The engineered protein of claim 1, wherein the three component proteins are the blue fluorescent protein, SERCA2a, and the yellow fluorescent protein, the blue fluorescent protein is fused to the n-terminus of SERCA in the A domain and the yellow fluorescent protein is fused to the N domain.

3. The engineered protein of claim 1, wherein the three component proteins are the blue fluorescent protein, SERCA2a, and the yellow fluorescent protein, the blue fluorescent protein is fused to the n-terminus of SERCA in the A domain and the yellow fluorescent protein is fused to the P domain.

4. The engineered protein of claim 1, wherein the three component proteins are the blue fluorescent protein, SERCA2a, and the yellow fluorescent protein, the blue fluorescent protein is fused to the n-terminus of SERCA in the A domain and the yellow fluorescent protein is fused to the TM domain.

5. The engineered protein of claim 1, wherein the three component proteins are the red fluorescent protein, SERCA, and the green fluorescent protein, the red fluorescent protein is fused to the n-terminus of SERCA in the A domain and the green fluorescent protein is fused to the N domain.

6. The engineered protein of claim 1, wherein the engineered protein utilizes only genetically encoded fluorophores.

7. The engineered protein of claim 1, wherein the engineered protein is encoded by a single plasmid.

8. A method of using the engineered protein of claim 1, the method comprising reporting of a structural state of the engineered protein during normal function.

9. The method of claim 8, wherein the reporting step comprises performing quantification of fluorescence resonance energy transfer (FRET) to observe rates of structure transitions under various physiological conditions.

10. The method of claim 8, further comprising the step of using the engineered protein as a targeted calcium sensor.

11. The method of claim 8, wherein the reporting step comprises measuring localized release of calcium from a sarco(endo)plasmic reticulum with high spatial resolution.

12. The method of claim 8, further comprising the step of using the engineered protein as a fluorescent sensor for high-throughput screening of drug libraries.

13. A method of determining SERCA activity for optimization of cardiac function, the method comprising resolving structure changes of a two-color SERCA construct comprising three component proteins fused together, wherein the two-color SERCA construct is catalytically active and able to pump calcium.

14. The method of claim 13, wherein the three component proteins are a blue fluorescent protein, SERCA2a, and a yellow fluorescent protein, the blue fluorescent protein is fused to the n-terminus of SERCA in the A domain, and the yellow fluorescent protein is fused to the N domain.

15. The method of claim 13, wherein the three component proteins are a blue fluorescent protein, SERCA2a, and a yellow fluorescent protein, the blue fluorescent protein is fused to the n-terminus of SERCA in the A domain, and the yellow fluorescent protein is fused to the P domain.

16. The method of claim 13, wherein the three component proteins are a blue fluorescent protein, SERCA2a, and a yellow fluorescent protein, the blue fluorescent protein is fused to the n-terminus of SERCA in the A domain, and the yellow fluorescent protein is fused to the TM domain.

17. The method of claim 13, wherein the three component proteins are a red fluorescent protein, SERCA, and a green fluorescent protein, the red fluorescent protein is fused to the n-terminus of SERCA in the A domain, and the green fluorescent protein is fused to the N domain.

18. The method of claim 13, further comprising the step of using the two-color SERCA construct as a fluorescent sensor for high-throughput screening of drug libraries.
